(19) United States
(12) Patent Application Publication
(10) Pub. No.: US 2023/0218865 A1
(43) Pub. Date: Jul. 13, 2023

(54) STABILIZER FOR AN INSERTED CATHETER AND METHOD FOR INSTALLING THE STABILIZER

(71) Applicant: Brian D. Worley, Tulsa, OK (US)

(72) Inventor: Brian D. Worley, Tulsa, OK (US)

(21) Appl. No.: 18/122,561

(22) Filed: Mar. 16, 2023

Related U.S. Application Data
(63) Continuation of application No. 17/127,300, filed on Dec. 18, 2020.

(51) Int. Cl. A61M 25/02

(52) U.S. Cl. CPC A61M 25/02; A61M 2025/0253; A61M 2025/0273

(57) ABSTRACT

A catheter stabilizer and a method for installing the stabilizer are especially suited to retaining a catheter in its position of insertion into the body of a patient. The stabilizer is a laminate of clear film atop double-backed adhesive preferably atop peel-off release paper. The double-backed adhesive defines a viewing window through the stabilizer. The stabilizer and method reduce preparation-of-materials steps, eliminate performance of arbitrary, awkward or inconsistently-performable installation steps, result in predictably effective catheter-to-patient connections, mitigate against the likelihoods of migration of the catheter out of the incision or expansion of the length of the incision during and after installation, create a sterile environment to the area surrounding the point of penetration of the catheter into the anatomy, extend the effective life of the installation, reduce the risks of complications and costs associated with replacement of a catheter and enable a rapid and sound installation.

### CROSS REFERENCE TO RELATED APPLICATIONS

This application is a Continuation of U.S. Pat. Application No. 17/127,300 titled “STABILIZER FOR AN INSERTED CATHETER AND METHOD FOR INSTALLING THE STABILIZER,” filed Dec. 18, 2020, the contents of which are hereby incorporated by reference.

### BACKGROUND OF THE INVENTION

This invention relates generally to the use of catheters and more particularly concerns the materials and methods used to retain an inserted catheter in a physically stable position in the anatomy of a patient.

Different surfaces of the human anatomy involve different motions, contours and textures that impact their reliability as a support for medical appliances and devices. For example, catheters are used to administer healing fluids to, or to evacuate damaging fluids from, various cavities in the human anatomy. Each use involves a different point of penetration into the anatomy and each penetration point has a different set of surrounding conditions. For example, a patient’s neck has a greater range-of-motion, a smaller diameter and different body-orifice and hair proximities than a patient’s torso.

For decades, neither the materials nor the practices of catheter installation have changed significantly. Known procedures employ materials ill-suited to effective catheter installation and require performance of awkward inconsistently-performable steps in accomplishing the catheter installation.

Arbitrary lengths of adhesive tape are torn from a roll and applied to the patient’s skin in an arbitrary overlapping pattern to form a base adhered to a broad area surrounding the point of penetration of the catheter into the anatomy. The exposed portion of the catheter is extended radially away from the point of penetration across the base lengths of tape. At least one other arbitrary length of tape is torn from the roll and cut or torn part-way into its width to form a slit. The slit is aligned transverse to the overlapping pattern and straddled on the catheter at the point of penetration with the closed end of the slit against, or as close as possible to, the catheter outer wall at the point of penetration. The half-width of the tape that does not overlie the exposed portion of the catheter is adhered to the base set the alignment of the split tape. The other half-width of the tape is manipulated under the exposed portion of the catheter and adhered to the base with the slit as closed as possible. Additional lengths of tape are torn from the roll and adhered as a cover for the base, the portion of the exposed catheter lying on the base, and the area surrounding the point of entry. If more than one length of slit tape is used, they are normally applied with their slits arranged at different angles. For example, two such slits would be at approximately right angles to each other.

Any catheter installation requires an incision at the point of catheter penetration, resulting in leakage of body fluids. The above-described steps are generally accomplished using non-sterile off-the-shelf adhesive tape, risking compromise of the sterility of the area and the possibility of acquired infections. This is especially of concern in connection with neck and face related catheters because of the proximity to the mouth and its “biologically dirty” oral secretions. Also, any strip of adhesive tape, once applied, blocks all visual observation and monitoring of the covered area, including the location of length markings on the catheter during and after, and any evidence of leakage or infection after, the installation. The arbitrary positioning of the strips of tape during installation and the associated deficiencies in permanent and smooth adherence of the tape to the skin or to the surfaces of already applied tape compromises, to varying degrees, the quality, durability and effectiveness of the installation. Such deficiencies increase the likelihood of migration of the catheter into and out of, and the expansion of the incision at, the point of penetration. They also tend to reduce the effective life of the installation, increasing the risks of complication and the costs associated with the replacement of the catheter.

As a result of the materials and methods presently employed, the resulting catheter-to-anatomy attachment structure is imperfect, imprecise, inaccurate and inadequate.

It is, therefore, a primary object of the invention to provide a catheter stabilizer and a method for installing the stabilizer which are especially suited to retaining a catheter in its position of insertion into the body of a patient. It is also an object of the invention to provide a catheter stabilizer and a method for installing the stabilizer which reduce the preparation-of-materials steps required for installation. Another object of the invention is to provide a catheter stabilizer and a method for installing the stabilizer which eliminate the need for performance of arbitrary, awkward or inconsistently-performable installation steps. A further object of the invention is to provide a catheter stabilizer and a method for installing the stabilizer which result in predictably effective catheter-to-patient connections. Yet another object of the invention is to provide a catheter stabilizer and a method for installing the stabilizer which mitigate against the likelihoods of migration of the catheter out of the incision or expansion of the length of the incision during and after installation. Still another object of the invention is to provide a catheter stabilizer and a method for installing the stabilizer which create a sterile environment to the area surrounding the point of penetration of the catheter into the anatomy. Yet a further object of the invention is to provide a catheter stabilizer and a method for installing the stabilizer which extend the effective life of the installation, thereby reducing the risks of complications and costs associated with replacement of a catheter. And it is an object of the invention to provide a catheter stabilizer and a method for installing the stabilizer which enable a rapid and sound installation.

### SUMMARY OF THE INVENTION

In accordance with the invention, a stabilizer retains a catheter in an inserted condition into the anatomy of a patient. In this condition, the catheter extends radially from its penetration point and along the anatomy. The stabilizer is a laminate of a layer of clear film and a layer of double-backed adhesive. A slit divides the laminate into a main body and an integral independently manipulable wing. The layer of double-backed adhesive defines a window through the main body. The window is sized and oriented so that the point of penetration and a portion of the catheter radially extending from the point of penetration are visible when the layer of double-backed adhesive is adhered to the anatomy with the manipulable wing extending transversely under the radially extending portion of the catheter. Preferably, the closed end of the slit defines an over-under aperture securing the catheter in place when the main body and the manipulable wing are adhered to the anatomy. Preferably, the laminate has a layer of peel-off release paper shielding an exposed face of the layer of double-backed adhesive prior to the adhering of the laminate to the anatomy.

For laminates having a front layer of clear film, an intermediate layer of double-backed adhesive and a rear layer of release papers, a first release paper covers the main body to a reference plane extending across the main body parallel to the slit and a second release paper covers the manipulable wing and the remainder of the main body to the reference plane. The window defined through the main body by the intermediate layer of double-backed adhesive is sized and oriented to permit the point of penetration and a portion of the catheter extending radially from the point of penetration to be visible when the layer of double-backed adhesive is adhered to the anatomy with the manipulable wing extending transversely under the radially extending portion of the catheter.

Preferably, the laminate has a generally rectangular outer perimeter with long and short edges and the slit extends from approximately a quarter-point of one of the long edges toward an opposite edge of the laminate. Preferably, the laminate has lengthwise and widthwise reference planes respectively extending across and perpendicular to the laminate and dividing the laminate into quadrants. Preferably, the slit extends beyond the lengthwise reference plane to define the independently manipulable wing. Preferably, the window defined by the layer of double-backed adhesive is aligned on the lengthwise reference plane and extends between the slit and a distal short side of the laminate. Preferably, for the three layer laminate, the first release paper has a length extending from the distal short edge of the laminate to the widthwise reference plane and the second release paper has a length sufficient to extend from a proximal short edge of the laminate to the widthwise reference plane. Also preferably, each release paper has a fold coincident with the widthwise reference plane and extending beyond the fold to form a flap of sufficient length to be fingertip gripped, the folds being coincident with the widthwise reference plane. The window may overlap the widthwise reference plane and the flaps when the flaps are folded back against their respective release papers.

Also in accordance with the invention, in the method for installing the stabilizer, the stabilizer is aligned approximately on a vertical lengthwise plane through an exposed portion of the catheter extending radially from the point of penetration toward a closed end of the slit. The aligned stabilizer is simultaneously smoothed and pressed to adhere a main body portion of the laminate against an underlying portion of the anatomy. The independently manipulable wing of the laminate is slipped under the catheter with the catheter seated in the closed end of the slit. The unadhered portions of the stabilizer are then simultaneously smoothed and pressed to adhere the unadhered portion of the main body of the laminate and the independently manipulable integral wing of the laminate against underlying portions of the anatomy and against the exposed portion of the catheter. If the adhesive layer has a window through the main body, as the stabilizer is aligned the window is also aligned to display the point of penetration of the catheter into the anatomy of the patient and the exposed portion of the catheter radially extending from the point of penetration along the vertical lengthwise plane toward a closed end of the slit.

If the adhesive layer has a window through the main body and the laminate includes a rear layer of release papers, the stabilizer is aligned approximately on a vertical lengthwise plane through the exposed radially extending portion of the catheter with the window positioned to display the point of penetration and the exposed portion of the catheter extending radially from the point of penetration toward a closed end of the slit. A first release paper of the releasable paper layer is peeled to reveal the double backed adhesive lying under the first release paper and to at least partly open the window so as to display at least the point of penetration of the catheter and a portion of the exposed portion of the catheter radially extending from the point of penetration. The aligned stabilizer is simultaneously smoothed and pressed to adhere the first-revealed double-backed adhesive against an underlying portion of the anatomy. The independently manipulable wing of the laminate is slipped under the catheter with the catheter seated in the closed end of the slit. A second release paper of the releasable paper layer is peeled to reveal the double-backed adhesive lying under the second release paper and to fully open the window to display more of the exposed portion of the catheter extending radially from the point of penetration. The unadhered portions of the stabilizer are then simultaneously smoothed and pressed to adhere the second revealed double-backed adhesive against an underlying portion of the anatomy and against the exposed portion of the catheter to complete the installation of the stabilizer.

### BRIEF DESCRIPTION OF THE DRAWINGS

Other objects and advantages of the invention will become apparent upon reading the following detailed description and upon reference to the drawings in which:

FIG. 1 is a front view of a catheter stabilizer in accordance with the invention;

FIG. 2 is a right edge view of the catheter stabilizer of FIG. 1;

FIG. 3 is a rear view of the catheter stabilizer of FIG. 1;

FIG. 4 is a front view of the front cover film of the catheter stabilizer of FIG. 1;

FIG. 5 is a rear view of the double-backed adhesive of the catheter stabilizer of FIG. 1;

FIG. 6 is a front view of a first release paper of the catheter stabilizer of FIG. 1;

FIG. 7 is a right edge view of the first release paper of FIG. 6;

FIG. 8 is a front view of a second release paper of the catheter stabilizer of FIG. 1;

FIG. 9 is a right edge view of the second release paper of FIG. 8;

FIG. 10 is a perspective view of the catheter stabilizer of FIG. 1;

FIG. 11 is a perspective view illustrating the catheter stabilizer of FIG. 1 ready for installation;

FIG. 12 is a perspective view illustrating a catheter inserted into and extended across a patient’s anatomy in preparation for installation of the catheter stabilizer of FIG. 11;

FIG. 13 is a perspective view illustrating the peeling of the first release paper of FIG. 6 from the catheter stabilizer of FIG. 11;

FIG. 14 is a perspective view illustrating the first revealed portion of the double-backed adhesive being applied to the base of the catheter stabilizer of FIG. 11;

FIG. 15 is a perspective view illustrating the peeling of the second release paper of FIG. 8 from the catheter stabilizer of FIG. 11; and

FIG. 16 is a perspective view illustrating the second revealed portion of the double-backed adhesive applied to the base of the catheter stabilizer of FIG. 11, completing the installation of the catheter stabilizer.

While the invention will be described in connection with a preferred embodiment and method thereof, it will be understood that it is not intended to limit the invention to that embodiment or method or to the details of the construction or arrangement of parts illustrated in the accompanying drawings.

### DETAILED DESCRIPTION

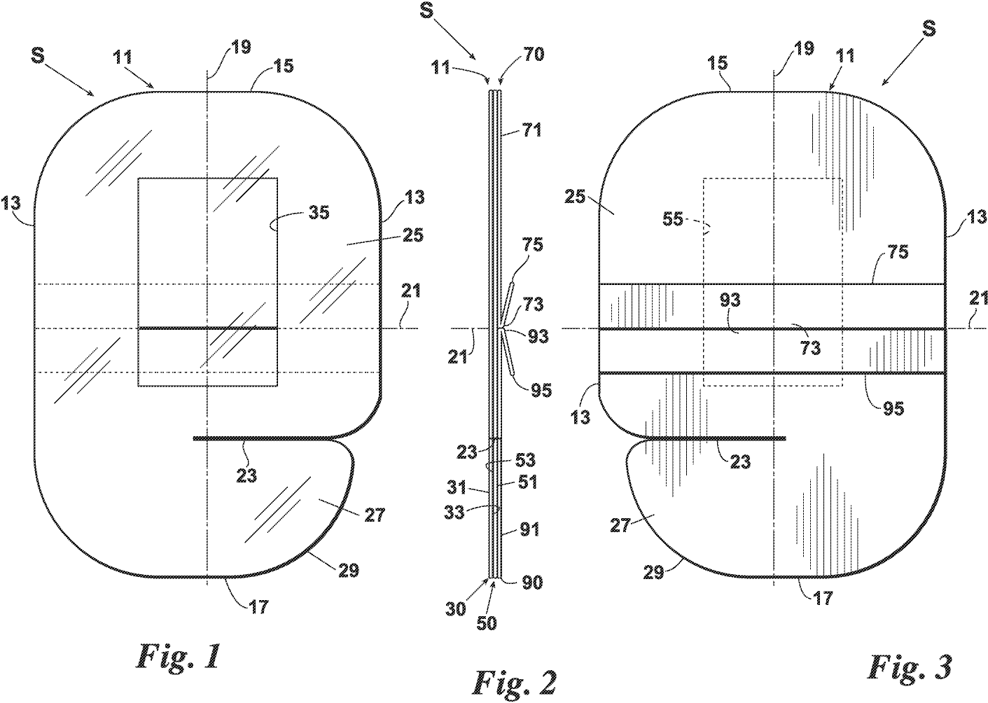

Looking at FIGS. 1-16, a stabilizer S in accordance with the invention is described in conjunction with retaining a catheter C in a stable position in the anatomy A of a patient. Catheters include chest tubes and dialysis catheters, central lines in the subclavian, internal jugular, and femoral veins or arterial lines in the radial, ulnar, and femoral arteries. The shape, composition and installation of the stabilizer S remain unchanged, but the stabilizer S is sized according to its use.

As seen in FIGS. 1-3, the stabilizer S is a lamination 10 of a front layer of clear film 30, an intermediate layer of double-backed adhesive 50 and a rear layer of side-by-side release papers 70/90. Preferably, the clear film 30 is polyurethane. Preferably, and as shown, the outer perimeter 11 of the lamination 10 is generally rectangular with rounded corners. It has long edges 13 of equal length, a short edge 15 and a shorter edge 17.

For purposes of further description, lengthwise and widthwise reference planes 19 and 21, respectively, are displayed across and perpendicular to the lamination 10, approximately dividing the lamination 10 into quadrants. A slit 23 extends from approximately a quarter-point of one of the long edges 13 toward, but short of, the opposite long edge 13 of the lamination 10. The slit 23 extends slightly beyond the lengthwise reference plane 19. Preferably, and as shown, the outer perimeter 11 of the lamination 10 rounds into the slit 23. The slit 23 divides the lamination 10 into a main body 25 and an integral independently manipulable wing 27 with a rounded corner 29 at the shorter edge 17 of the lamination 10.

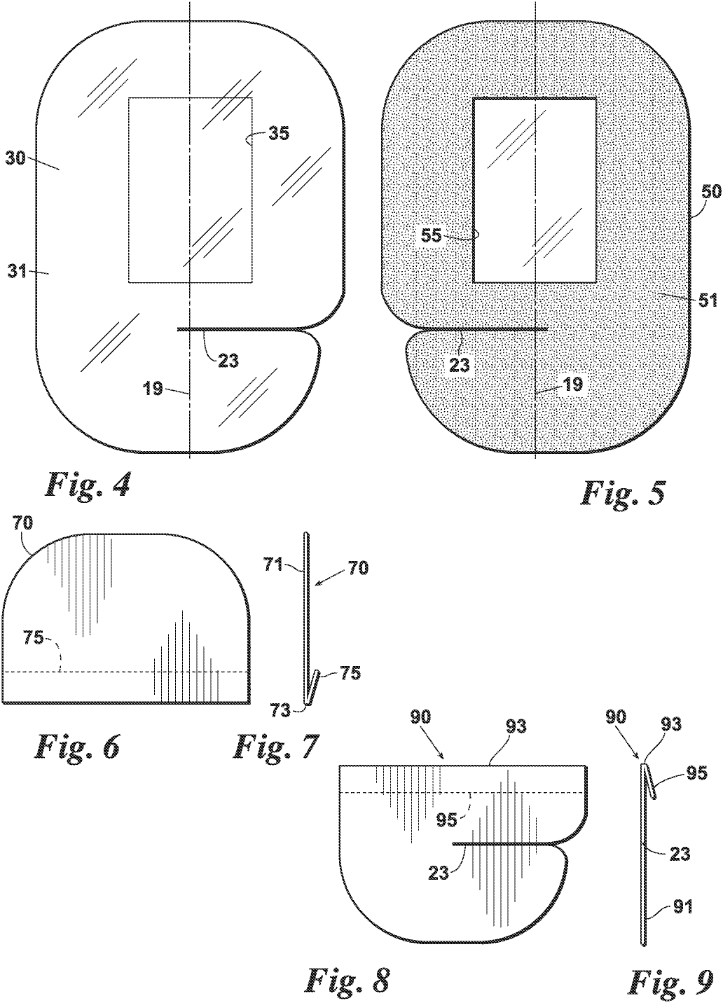

Moving on to side-by-side FIGS. 4 and 5, the front face 31 of the clear film 30 and the rear face 51 of the double-backed adhesive 50 are mirror images. Looking again at FIG. 2, the front face 53 of the double-backed adhesive 50 is applied to the rear face 33 of the clear film 30. Returning to FIGS. 4 and 5, preferably, and as shown, the double-backed adhesive 50 covers all of the rear face 33 of the clear film 30 except for a rectangular window 55 through the double-backed adhesive 50. Preferably, and as shown, the window 55 is symmetrically aligned on the lengthwise reference plane 19 and extends between the slit 23 and the short edge 15 of the lamination 10. When the front face 53 of the double-backed adhesive 50 is applied to the rear face 33 of the clear film 30, the rectangular window 55 through the double-backed adhesive 50 is coincident with a phantom rectangular delineation 35 of the window 55 on the clear film 30.

Looking at FIGS. 6-7, first release paper 70 has a length 71 sufficient to extend from the short edge 15 of the lamination 10 to the widthwise reference plane 21. Preferably, and as shown, the first release paper 70 has a fold 73 coincident with the widthwise reference plane 19 and extends beyond the fold 73 to form a flap 75 of sufficient length to provide a fingertip grip.

Looking at FIGS. 8-9, the second release paper 90 has a length 91 sufficient to extend from the shorter edge 17 of the lamination 10 to the widthwise reference plane 21. Preferably, and as shown, the release paper 90 has a fold 93 coincident with the widthwise reference plane 21 and extends beyond the fold 93 to form a flap 95 of sufficient length to provide a fingertip grip.

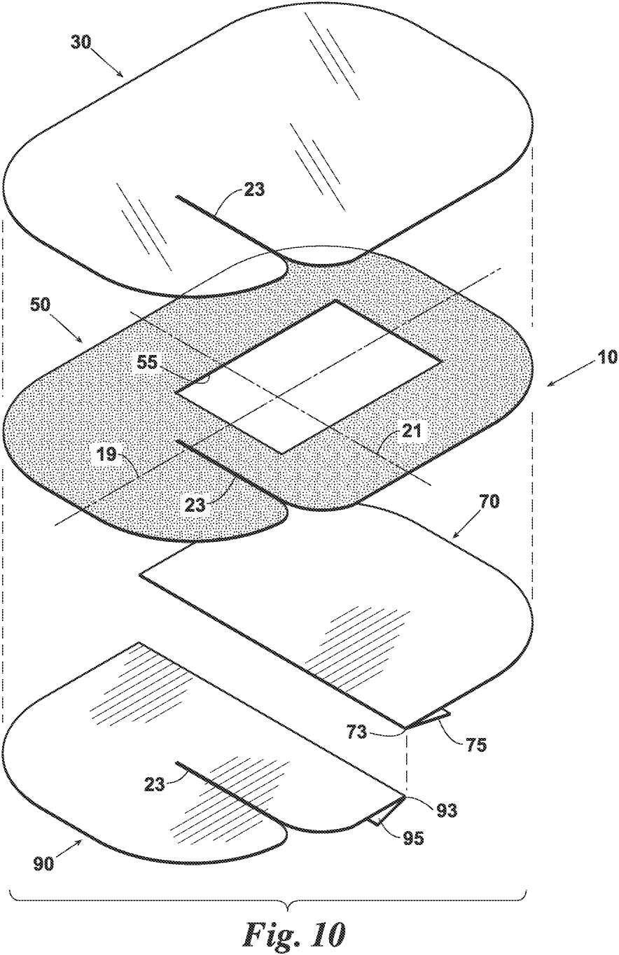

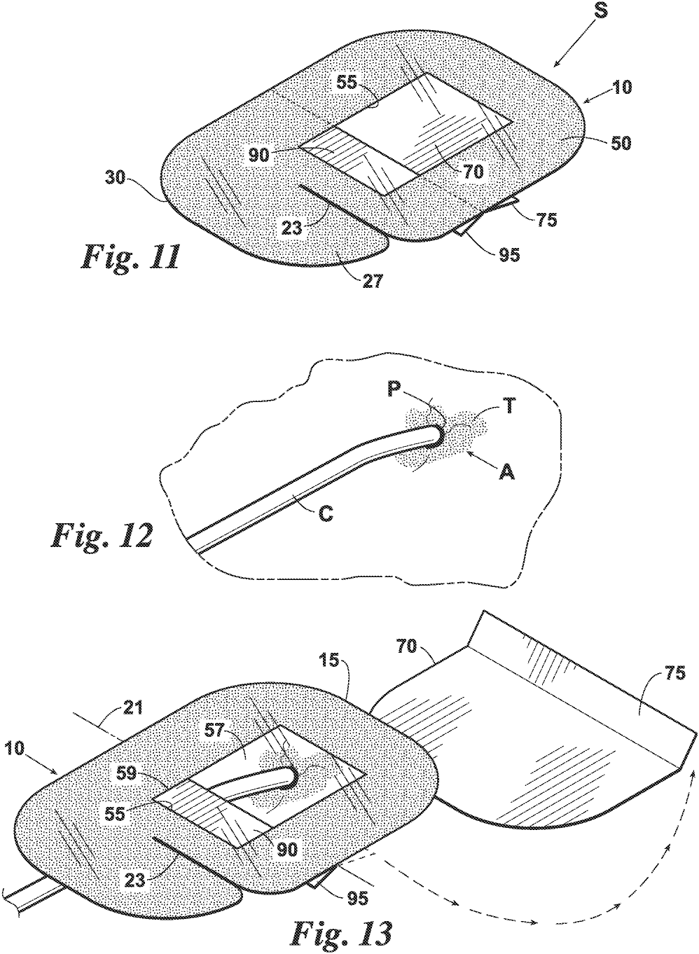

FIG. 10 illustrates the preferred relationships of the layers of the laminate 10 as being separated from each other but aligned as parts of the laminate 10. However, FIG. 10 is not an assembly drawing. The laminate 10 may be formed using steps differing from the steps suggested by the drawing. Rather, FIG. 10 is intended to further illustrate the preferred inter-relationships of the front layer of clear film 30, the intermediate layer of double-backed adhesive 50 including the window 55, the rear layer of first and second side-by-side release papers 70/90, the lengthwise and widthwise reference planes 19 and 21 of the laminate 10 and the slit 23 in the laminate 10. Furthermore, in FIG. 10, the first and second release papers 70/90 are shown in a separated condition rather than in the side-by-side condition of the laminate 10. And, looking at FIGS. 1-3 and 10, when the flaps 75 and 95 are folded back against their respective first and second release papers 70/90, the window 55 overlaps the widthwise reference plane 21 and the flaps 75/95, the folds 73 and 93 are abutting and the flaps 75 and 95 do not come into contact with the double-backed adhesive 50.

FIG. 11 illustrates the stabilizer S oriented in a ready to install state. FIG. 12 illustrates the catheter C inserted into the anatomy A, particularly into the torso T at a point of penetration P, in an orientation and a condition ready for installation of the stabilizer S. Turning to FIGS. 13-16, the method for installing the stabilizer S, as seen in FIG. 11, on the patient’s anatomy A with the catheter C in its inserted condition, as seen in FIG. 12, can be understood.

In FIG. 13, the flap 75 of the first release paper 70 has been gripped and peeled from the widthwise reference plane 21 until the release paper 70 is entirely detached from the lamination 10. The rear face 51 of the double-backed adhesive 50 has been revealed entirely from the widthwise reference plane 21 to the short edge 15 of the lamination 10 and a substantial portion 57 of the window 55 has been “opened.” A smaller portion 59 of the window 55 remains “closed” by the second release paper 90.

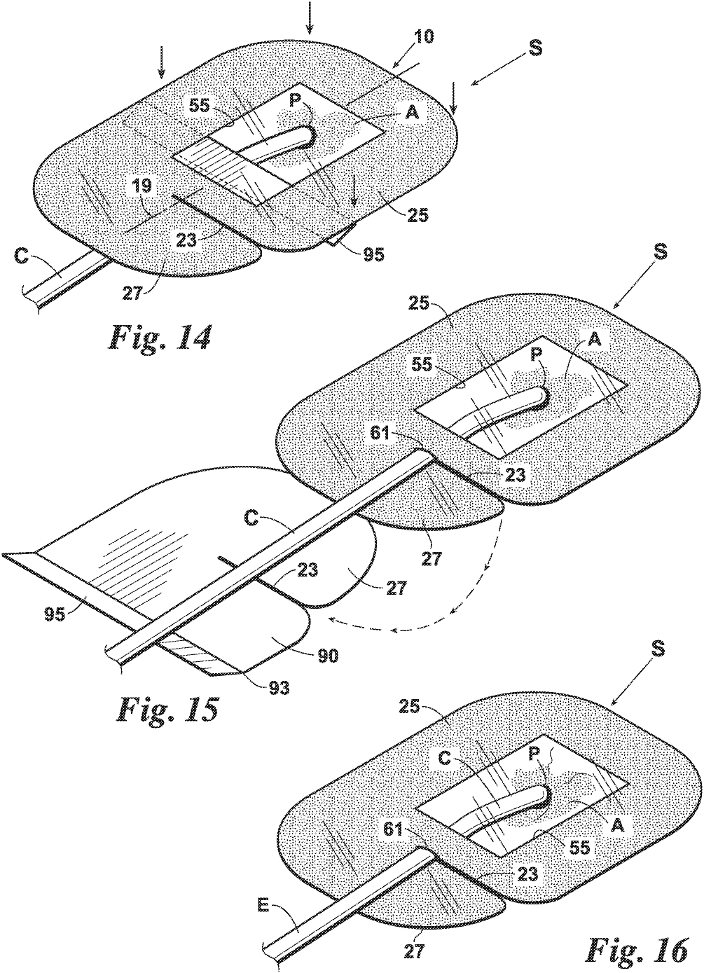

As seen in FIG. 14, the longitudinal axis of the catheter C is in approximate alignment with the vertical lengthwise plane 19 of the lamination 10. The point of penetration P of the catheter C into the anatomy A is approximately centered in the window 55. The revealed portion of the rear face 51 of the double-backed adhesive 50 is lowered, smoothed and pressed over the catheter C and against the anatomy A of the patient to secure the corresponding portion of the stabilizer S in place and to partially secure the catheter C in proper alignment with the stabilizer S.

Moving on to FIG. 15, with a substantial portion of the main body 25 of the stabilizer S secured in place to hold the catheter C in proper alignment, the flap 95 of the second release paper 90 is peeled to reveal the remainder of the rear face 51 of the double-backed adhesive 50. As it is being peeled, the newly revealed portion of the main body 25 is smoothed and pressed against the underlying portion of the anatomy A and the catheter C up to the closed end of the slit 23. The catheter C is then lifted against the portion of the main body 25 extending between the window 55 and the slit 23 that bridges is adhered to the catheter C. The independently manipulable wing 27 of the laminate 10 is then slipped under the catheter C and smoothed and pressed against the anatomy A, locking the catheter C in the over-under aperture 61 created by the bridging portion of the main body 25 adhered to the catheter C, the underlying portion of the wing 27 adhered to the anatomy A and the substantial reclosing of the slit 23.

The final condition resulting at the completion of the installation of the stabilizer S is seen in FIG. 16. The point of penetration P of the catheter C into the anatomy A and the alignment of the catheter C from the point of penetration P to proximate the aperture 61 can be observed through the window 55. The alignment of the catheter C from the aperture 61 along the extending portion E of the catheter C is entirely exposed.

Preferably, the stabilizer is sterilized and packaged to decrease the risk that infection be acquired because of the installation. The combination of the clear cover and the window through the double-backed adhesive allows visual observation and monitoring of the area surrounding the incision site during and after installation of the stabilizer, including but not limited to the position of length markings on the catheter during and after installation of the stabilizer and evidence of leakage or infection after installation of the stabilizer. The stabilizer mitigates against the likelihood of migration of the catheter out of, and expansion of the incision at, the point of penetration. The effectiveness of the stabilizer allows the catheter to be left in longer, decreasing the risks of complication and the costs associated with replacement of the catheter.

Thus, it is apparent that there has been provided, in accordance with the invention, a stabilizer and a method for installing the stabilizer to retain a catheter in an inserted condition in the anatomy of a patient that fully satisfies the objects, aims and advantages set forth above. While the invention has been described in conjunction with a specific embodiment thereof, it is evident that many alternatives, modifications and variations will be apparent to those skilled in the art and in light of the foregoing description. Accordingly, it is intended to embrace all such alternatives, modifications and variations as fall within the spirit of the appended claims.

What is claimed is:

1. A stabilizer for retaining a catheter tube inserted at a penetration point into the anatomy of a patient and extending radially from the penetration point along the anatomy, the stabilizer comprising:
a laminate having a layer of clear film atop a layer of double-backed adhesive;
a slit dividing said laminate into a main body and an integral independently manipulable wing; and
said layer of said double-backed adhesive having a window therethrough to permit the point of penetration and the portion of the catheter tube extending radially along the anatomy from the point of penetration to be visible through said layer of clear film when said layer of said double-backed adhesive is adhered to the anatomy with said integral independently manipulable wing extending transversely through said slit and under the portion of the catheter tube extending radially along the anatomy.

2. A stabilizer according to claim 1, a closed end of said slit defining an over-under aperture securing the catheter tube in place on the anatomy when said main body and said integral independently manipulable wing are adhered to the anatomy.

3. A stabilizer according to claim 1, said laminate further comprising a layer of peel-off release paper shielding an exposed face of said layer of double-backed adhesive of said laminate prior to the adhering of said laminate to the anatomy.

4. A stabilizer for retaining a catheter tube inserted at a point of penetration into the anatomy of a patient and extending radially from the point of penetration along the anatomy, the stabilizer comprising:
a laminate having a front layer of clear film, an intermediate layer of double-backed adhesive and a rear layer of release papers;
a slit dividing said laminate into a main body and an integral independently manipulable wing;
a first of said release papers covering said main body to a reference plane extending across said main body parallel to said slit;
a second of said release papers covering said integral independently manipulable wing and said main body to said reference plane; and
said intermediate layer of said double-backed adhesive having a window therethrough sized and oriented to permit the point of penetration and a portion of the catheter tube extending radially from the point of penetration along the anatomy to be visible through said front layer of clear film when said intermediate layer of double-backed adhesive is adhered to the anatomy with said integral independently manipulable wing extending transversely through said slit and under the portion of the catheter tube extending radially along the anatomy.

5. A stabilizer according to claim 4, said laminate having a generally rectangular outer perimeter with long and short edges and said slit extending from approximately a quarter-point of one of said long edges toward a said opposite long edge of said laminate.

6. A stabilizer according to claim 5, said laminate having lengthwise and widthwise reference planes, respectively, extending across and perpendicular to said laminate and dividing said laminate into quadrants, and said slit extending beyond said lengthwise reference plane to define said independently manipulable wing.

7. A stabilizer according to claim 6, said intermediate layer of said double-backed adhesive having said window aligned on said lengthwise reference plane and extending between said slit and a distal said short side of said laminate.

8. A stabilizer according to claim 6, said first said release paper having a length extending from said distal said short edge of said laminate to said widthwise reference plane and said second said release paper having a length sufficient to extend from a proximal said short edge of said laminate to said widthwise reference plane.

9. A stabilizer according to claim 8, each said release paper having a fold coincident with said widthwise reference plane and extending beyond said fold to form a flap of sufficient length to be fingertip gripped, said folds of said release paper being coincident with said widthwise reference plane.

10. A stabilizer according to claim 9, said window overlapping said widthwise reference plane and said flaps when said flaps are folded back against their respective said release papers.

11. A method for installing a stabilizer to retain a catheter tube in an inserted condition at a point of penetration into the anatomy of a patient, the stabilizer being a laminate of front clear film and rear double-backed adhesive layers, and a slit dividing the laminate into a main body and an independently manipulable integral wing, the method comprising the steps of:
aligning the stabilizer approximately on a vertical lengthwise plane through the catheter tube with an exposed portion of the catheter tube extending radially from the point of penetration along the vertical lengthwise plane toward a closed end of the slit;
simultaneously smoothing and pressing the aligned stabilizer to adhere a portion of the main body of the laminate against an underlying portion of the anatomy;
slipping the independently manipulable wing of the laminate into transverse alignment under the catheter tube with the catheter tube seated in the closed end of the slit; and
simultaneously smoothing and pressing the unadhered portions of the stabilizer to adhere the unadhered portion of the main body of the laminate and the independently manipulable integral wing of the laminate against underlying portions of the anatomy and the exposed portion of the catheter tube.

12. A method according to claim 11, the adhesive layer having a window through the main body and the step of aligning further comprising the window displaying the point of penetration of the catheter tube into the anatomy of the patient and an exposed portion of the catheter tube extending radially from the point of penetration along the vertical lengthwise plane toward the closed end of the slit.

13. A method for installing a stabilizer to retain a catheter tube in an inserted condition at a point of penetration into the anatomy of a patient, the stabilizer having laminate front clear film, intermediate double-backed adhesive and rear releasable paper layers and a slit dividing the laminate into a main body and an independently manipulable integral wing, the adhesive layer having a window through the main body, the method comprising the steps of:
aligning the stabilizer approximately on a vertical lengthwise plane through the catheter tube with the window positioned to display the point of penetration of the catheter tube into the anatomy of the patient and an exposed portion of the catheter tube extending radially from the point of penetration toward a closed end of the slit;
peeling a first release paper of the releasable paper layer, revealing the double backed adhesive lying under the first release paper and at least partly opening the window so as to display at least the point of penetration of the catheter tube into the anatomy of the patient and a portion of the exposed portion of the catheter tube extending radially from the point of penetration;
simultaneously smoothing and pressing the aligned stabilizer to adhere the first-revealed double backed adhesive against an underlying portion of the anatomy;
slipping the independently manipulable wing of the laminate transversely under the catheter tube with the catheter tube seated in the closed end of the slit;
peeling a second release paper of the releasable paper layer, revealing the double backed adhesive lying under the second release paper and fully opening the window to display more of the exposed portion of the catheter tube extending radially from the point of penetration; and
simultaneously smoothing and pressing the unadhered portions of the stabilizer to adhere the second-revealed double backed adhesive against an underlying portion of the anatomy and the exposed portion of the catheter tube.

14. A stabilizer for retaining a catheter tube inserted at a point of penetration into the anatomy of a patient and extending along the patient’s anatomy radially from the point of penetration, the stabilizer comprising:
a laminate comprising a layer of clear film atop a layer of double-backed adhesive;
a slit dividing said laminate into a main body and an integral independently manipulable wing; and
a window defined within said layer of double-backed adhesive;
said window permitting a portion of the catheter tube between the point of penetration and a point on the catheter tube closer to said slit than the point of penetration to be visible through said layer of clear film when said layer of said double-backed adhesive is adhered to the anatomy with said integral independently manipulable wing extending transversely through said slit and under the portion of the catheter tube extending radially along the anatomy.

15. A stabilizer for retaining a catheter tube inserted at a point of penetration into the anatomy of a patient and extending along the patient’s anatomy radially from the point of penetration, the stabilizer comprising:
a laminate comprising a front layer of clear film, an intermediate layer of double-backed adhesive and a rear layer of release papers;
a slit dividing said laminate into a main body and an integral independently manipulable wing;
a first said rear layer release paper covering said main body to a reference plane extending across said main body parallel to said slit;
a second said rear layer release paper covering said integral independently manipulable wing and said main body to said reference plane; and
a window defined within said intermediate layer of double-backed adhesive;
said window permitting a portion of the catheter tube between the point of penetration and a point on the catheter tube closer to said slit than the point of penetration to be visible through said front layer of clear film when said first and second rear layer release papers are removed from said intermediate layer of double-backed adhesive and said intermediate layer of double-backed adhesive is adhered to the anatomy with said integral independently manipulable wing extending transversely through said slit and under the portion of the catheter tube extending radially along the anatomy.
